SEQUENCE LISTING (26 sequences)

SEQ ID NO: 1 - DNA, 24 bp, HUMAN
gcggccgctaaggtgaggcaggtg

SEQ ID NO: 2 - DNA, 20 bp, HUMAN
aagcttgccggccgtcgcac

SEQ ID NO: 3 - DNA, 29 bp, HUMAN
cccctgcagggagtgcatccgccccaacc

SEQ ID NO: 4 - DNA, 26 bp, HUMAN
gggaagcttcagtagcaggtgccagc

SEQ ID NO: 5 - DNA, 25 bp, HUMAN
gcggccgccagcacagggagggagg

SEQ ID NO: 6 - DNA, 18 bp, HUMAN
gggctctgcagagagaag

SEQ ID NO: 7 - DNA, 32 bp, HUMAN
gcgctgcagacaaaactcacacatgcccaccg

SEQ ID NO: 8 - DNA, 23 bp, HUMAN
gcgaagcttgccggccgtcgcac

SEQ ID NO: 9 - DNA, 28 bp, HUMAN
gcggcggccgccaaaccctcccagcagg

SEQ ID NO: 10 - DNA, 27 bp, HUMAN
cgcgaattcgcctggttgagcgctagc

SEQ ID NO: 11 - DNA, 34 bp, HUMAN
cgcgcgcggccgctcgaggccacaatgaaccggg

SEQ ID NO: 12 - DNA, 42 bp, HUMAN
gcggcggccgcacttacctagcaccacgatgtctattttgaa

SEQ ID NO: 13 - DNA, 81 bp, HUMAN
ctcgagatggagtttgggctgagctggctttttcttgtggctattttaaaaggtgtccagtgtgaggtgcagctggtggag

SEQ ID NO: 14 - DNA, 36 bp, HUMAN
gcggccgcacttacctaggacggtcagcttggtccc

SEQ ID NO: 15 - DNA, 51 bp, HUMAN
gcggcggccgcactcacctgaggagacggtgaccagggtaccttggcccca

SEQ ID NO: 16 - DNA, 87 bp, HUMAN
gcgctcgagatggcctggacccctctctggctcactctcctcactctttgcataggttctgtggtttcttctgagctgactcaggac

SEQ ID NO: 17 - protein, 18 aa, HUMAN
GSTSGSGKPGSGEGSTKG

SEQ ID NO: 18 - DNA, 54 bp, HUMAN
ggctctacttccggttcaggaaagcccgggagtggtgaaggtagcactaaaggt

SEQ ID NO: 19 - DNA, 60 bp, HUMAN
cgccccgggctttcctgaaccggaagtagagccagctagcaccacgatgtctattttgaa

SEQ ID NO: 20 - DNA, 51 bp, HUMAN
gcgcccgggagtggtgaaggtagcactaaaggtgaggtgcagctggtggag

SEQ ID NO: 21 - DNA, 23 bp, HUMAN
gcgctggagatggagttttgggc

SEQ ID NO: 22 - DNA, 51 bp, HUMAN
cgccccgggctttcctgaaccggaagtagagcctgaggagacggtgaccag

SEQ ID NO: 23 - DNA, 54 bp, HUMAN
gcccccgggagtggtgaaggtagcactaaaggtcagggaaagaaagtggtgctg

SEQ ID NO: 24 - DNA, 609 bp, HUMAN
atgaaccggggagtcccttttaggcacttgcttctggtgctgcaactggcgctcctcccagcagccactcagggaaagaaagtggtgctgggcaaaaaaggggatacagtggaactgacctgtacagcttcccagaagaagagcatacaattccactggaaaaactccaaccagataaagattctgggaaatcagggctccttcttaactaaaggtccatccaagctgaatgatcgcgctgactcaagaagaagcctttgggaccaaggaaacttccccctgatcatcaagaatcttaagatagaagactcagatacttacatctgtgaagtggaggaccagaaggaggaggtgcaattgctagtgttcggattgactgccaactctgacacccacctgcttcaggggcagagcctgaccctgaccttggagagcccccctggtagtagcccctcagtgcaatgtaggagtccaaggggtaaaaacatacagggggggaagaccctctccgtgtctcagctggagctccaggatagtggcacctggacatgcactgtcttgcagaaccagaagaaggtggagttcaaaatagacatcgtggtgctagct

SEQ ID NO: 25 - DNA, 351 bp, HUMAN
gaggtgcagctggtggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcaagacggcggcatcgtgcttttgactattggggccaaggtaccctggtcaccgtctcctca

SEQ ID NO: 26 - DNA, 899 bp, HUMAN
gacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

(19) United States
(12) Patent Application Publication
(10) Pub. No.: US 2004/0110922 A1
(43) Pub. Date: Jun. 10, 2004

(54) Chimeric proteins for prevention and treatment of hiv infection

(76) Inventors: Gideon Gross, Korazim (IL); Ronit Meyuhas, Hadassah (IL)

Correspondence Address: BROWDY AND NEIMARK, P.L.L.C., 624 NINTH STREET, NW, SUITE 300, WASHINGTON, DC, 20001-5303, US

(21) Appl. No.: 10/466,836

(22) PCT Filed: Jan. 22, 2002
(86) PCT No.: PCT/IL02/00060

(30) Foreign Application Priority Data
Jan. 22, 2001 (IL) 141023

(51) Int. Cl. C07K 16/46; C07K 14/16; C07H 21/04

(52) U.S. Cl. 530/350; 530/391.1; 435/69.1; 435/328; 435/320.1; 536/23.53

(57) ABSTRACT

Chimeric proteins are provided having binding specificity for at least two different sites, at least one site being on the HIV envelope glycoprotein gp120 and the other bite being either on said gp120 protein or on the extracellular portion of human CD4, said chimeric protein comprising: (a) a first binding region comprising a soluble extracellular portion of human CD4; (b) a second binding region comprising a variable region of an antibody heavy chain, preferably VH3; (c) a linker that connects (a) and (b); and, optionally, a portion of a constant region of an immunoglobulin chain. The chimeric proteins are useful for prevention and treatment of HIV infection.

### FIELD OF THE INVENTION

The present invention relates to nucleic acid molecules encoding chimeric proteins that bind either to two different and independent sites on the HIV envelope glycoprotein gp120 or to one site on gp120 and to one site on the extracellular portion of human CD4. These chimeric proteins are useful in the prevention and treatment of HIV infection.

### BACKGROUND OF THE INVENTION

Increasing the affinity of a molecule with a potential medical use for its ligand is most likely to improve its efficacy as a therapeutic drug or a diagnostic tool.

Protein molecules, encoded by cloned genes, are amenable to modifications through genetic engineering. Numerous studies have reported the enhancement of the affinity of proteins for their ligands using site-directed mutagenesis, introducing amino-acid substitutions into the ligand-binding site of the protein. Such products have been routinely shown to be superior to the wild-type molecules in exerting their biological functions. For example, the virus neutralizing ability of a human antibody to human immunodeficiency virus (HIV) was significantly improved after its in-vitro evolution which resulted in higher affinity for its viral antigen [Barbas, C. F. et al. (1994) Proc. Natl. Acad. Sci. USA 91:3809-3813]. A highly efficient and well-tuned machinery for enhancing affinities is manifested in the process of affinity maturation of antibodies for the eliciting antigen, which takes place in lymphoid organs during the development of an immune response.

Engrafting on the same polypeptide, via a peptide linker, two binding sites for distinct determinants on the ligand molecule, is another conceivable approach for augmenting binding affinities, which seems to have received much less attention by experimentalists. In order to perform this task, these determinants must not overlap, but are still to be situated close enough on the same ligand molecule. This should ensure a minimal distance between the two binding sites, and favor a 1:1 stoichiometry between the polypeptide and its ligand. A key parameter in governing the expected affinity enhancement is the rigidity of the bridge between the binding sites. A linker, which is too rigid, may prevent proper arrangement of all interacting moieties, but over flexibility may diminish the expected ‘anchor’ effect. Kinetically, simultaneous binding is expected to confer a significantly lower off-rate of binding compared with each of the separate interactions. Thermodinamically, the two linked binding sites can simply be regarded as a single, extended site, resulting in a significant gain in free energy of binding. This principle has been exemplified using two anti-hen egg lysozyme (HEL) monoclonal antibodies (MAbs), whose crystal structure in complex with the antigen had previously been solved [Neri, D., et al. (1995) J. Mol. Biol 246; 367-373]. The authors linked two single-chain Fv (scFv) structures, produced from these two MAbs on the same polypeptide via a hydrophilic flexible peptide linker. The affinity of the resultant molecule for HEL was shown to be 1-2 orders of magnitude higher than that of the stronger binder of the two single scFvs, depending on the method used for measurement. However, this system is of very limited physiological relevance, and it does not allow direct attribution of functional properties to the resulting higher affinity.

The first step in HIV infection is binding of the large subunit of the envelope glycoprotein (env) of HIV-1, known as gp120, to receptors on target cells. The major cellular receptor for HIV is the CD4 molecule [reviewed by Capon, D. J., & Ward, R. H. R., (1991) Annu. Rev. Immunol. 9:649-678]. Binding to gp120 is exclusively mediated by residues in the immunoglobulin (Ig) variable region (V)-like membrane-distal domain of the CD4 molecule designated V1 (or D1) [Kwong, P. D., et al. (1998) Nature 393:648-659], but the second domain, V2 (or D2), has also been shown to affect binding. The affinity between CD4 and gp120 has been estimated to be in the nanomolar range [Lasky, L., et al. (1987) Cell 50;975-985; Smith, D. H. et al. (1987) Science 328:1704-1707]. The three dimensional structure of the gp120 core has been determined [Kwong, P. D., et al. (1998) Nature 393:648-659; Wyatt, R., et al. (1998) Nature 393:705-711; Rizzuto, C. D., et al. (1998) Science 280:1949-1953]. CD4 interacts with a broad crevice-like area on gp120, formed by residues from several regions, with many contacts made with backbone atoms of gp120 amino acids other than their side chains.

Recombinant soluble CD4 (rsCD4), especially in the form of immunoligands (or immunoadhesins), either with the full extracellular portion (V1-V4 or D1-D4) or in the truncated V1+V2 (D1+D2) configuration, has received a lot of attention as a potential HIV neutralizing drug [Capon, D. J., & Ward, R. H. R., (1991) Annu. Rev. Immunol. 9;649-678 and references therein]. However, while proving effective with most HIV laboratory strains, much higher concentrations of rsCD4 were required to achieve the same effect with a panel of primary isolates of the virus [Daar, E. S., et al. (1990) Proc. Natl. Acad. Sci. USA 87:6574-6578]. The reason for this relative resistance is still not clear, but it is not attributed to lower affinity for CD4 [Turner, S., et al.(1992) Proc. Natl. Acad. Sci. USA 89:1335-1339]. Effective CD4-based HIV therapeutics are therefore likely to require significantly higher affinity for gp120 than native CD4.

International PCT Publication No. WO 00/55207 discloses a neutralizing bispecific fusion protein capable of binding to two sites on a target protein, particularly a gp-120-targeted fusion protein, comprising a first binding domain capable of binding to an inducing site on the target protein (gp120), thereby exposing an induced epitope, a second binding domain, which is capable of forming a neutralizing complex with an induced epitope of the target protein, and a linker connecting both domains. The first binding domain is composed of the soluble CD4 containing domains D1 and D2 (183 amino acid residues) and the second binding domain is composed of a single chain Fv portion of the human monoclonal antibody designated 17b, that binds to the CD4-induced epitope on gp120.

U.S. Pat. Nos. 6,004,781 and 6,117,656 disclose nucleic acid molecules encoding a fusion protein comprising a DNA sequence encoding amino acids 1-173 of CD4 and a DNA sequence encoding an immunoglobulin heavy or light chain, wherein the variable region of the immunoglobulin chain has been replaced with the sequence encoding amino acids 1-173 of CD4, whereby a nucleic acid encoding a fusion protein capable of being secreted is formed.

HIV gp120 was reported to bind to Ig molecules on human B cells bearing heavy chains with variable regions (VH) encoded by members of the VH3 gene family [Berberian, L., et al. (1993) Science 249:1588-1591]. This binding triggers activation of several percent of the total normal human B cell population, thus classifying HIV gp120 as a B cell superantigen (SA), and occurs similarly with nonimmune serum antibodies. Two publications have reported the identification of the VH3 binding site on gp120 [Goodlick, L., et al. (1995) J. Immunol. 155:5151-5159; Karray, S., & Zouali, M., (1997) Proc. Natl. Acad. Sci. USA 94:1356-1360], using synthetic gp120-derived peptides as binding inhibitors. From these works it stems that the border region between the third variable domain (V3) and the fourth conserved domain (C4) of gp120 is crucial for binding, with important contribution from the second conserved domain (C2).

The VH3 gene family comprises 19 of the 39 functional human VH genes [Matsuda, F., et al. (1998) J. Exp. Med. 188:2151-2162], but only a subset of these genes, including VH3-23 and VH3-30, have been shown to impart gp120 SA binding [Karray, S., et al. (1998) J. Immunol. 161:6681-6688]. Genetic analysis of SA-binders and non-binders has correlated binding with amino acid residues positioned primarily in the first and third framework regions (FR1 and FR3). Genetic manipulations of VH3-30 [Neshat, M. N., et al. (2000) Int. Immunol. 12:305-312] have attributed a role in SA binding to multiple discontinuous arrayed residues from hypervariable loop 1 (H1) and, to a lesser extent, to H2 and all three FRs. The conclusion from these two works is that amino acids contributing to gp120 SA binding reside along the whole domain. Neither the light chain of the antibody molecule nor H3 (equivalent to the third complementarity determining region, or CDR3) have been implicated in gp120 SA binding. A VH3 domain, encoded by either VH3-23 or VH3-30, is therefore a likely candidate to be linked to the membrane-distal portion of CD4, as argued above.

The V1 (or D1) domain of the mature human CD4 protein, constitutes residues 1-92 [Maddon, P. J. et al. (1985) Cell 42:93-104; see correction by Littman, D. R. et al. (1988) Cell 55:541]. Sequence analysis and determination of atomic structure [Wang, J. et al. (1990) Nature 348:411-418; Kwong, P. D. et al. (1991), Proc. Natl. Acad. Sci. (USA) 87:6423-6427; Sweet, R. W. et al. (1991) Curr. Opin. Biotech. 2:622-633] reveal that this domain consists of nine β strands arranged in a cylindrical shape, which is stabilized by an intra-domain disulfide bond. Topology and core elements in this structure resemble those of a variable domain of an antibody light chain (VL). Indeed, V1 loops, corresponding to CC′ and CDR3 loops in VL, which are involved in pairing with VH in an antibody molecule, are represented to a lesser degree. However, the actual interface between VL and VH consists primarily of the four strands C′—C—F-G [Chothia et al. (1985) J. Mol. Bio. 186:651-663], which can be identified in the V1 structure. Hence, possible association between V1 and an unpaired VH domain, through these four β strands, cannot be ruled out. As in the formation of an antibody Fv, dimerization of CD4 V1 domain with an antibody VH domain may induce re-positioning of V1 residues. Such a rearrangement may stabilize a certain conformation, which increases the binding affinity of CD4 for gp120.

### SUMMARY OF THE INVENTION

It has now been found, according to the present invention, that when a sequence of the variable region of an antibody heavy chain is fused with the extracellular portion of CD4, the capacity of the soluble CD4 to interact with gp 120 is enhanced and the interaction of the HIV virus with cellular CD4 can be thereby blocked more effectively than by soluble CD4 alone. 10 The present invention thus relates, in one aspect, to a nucleic acid molecule encoding a functional chimeric protein having binding specificity for at least two different sites, at least one site being on the HIV envelope glycoprotein gp120 and the other site being either on said gp120 protein or on the extracellular portion of human CD4, wherein the binding to said at least one site on said gp120 protein is independent of its binding to said other site on said gp 120 protein or on said extracellular portion of human CD4, said chimeric protein essentially comprising:

(a) a first binding region comprising a soluble extracellular portion of human CD4;

(b) a second binding region that comprises a variable region of an antibody heavy chain, that is capable of being attached to an adjacent and non-overlapping site on the said gp120 protein or to a site on said extracellular portion of human CD4, and is capable of increasing the capacity of the said extracellular portion of human CD4 to interact with gp120 and to block the interaction of HIV with membranal CD4; and

(c) a linker region that physically connects both binding regions (a) and (b).

The first binding region of the nucleic acid molecule encodes preferably the two extracellular membrane distal domains of CD4, V1 and V2, encoded by the DNA sequence substantially as denoted by SEQ ID NO:24, and the second binding lion encodes preferably a variable region of an antibody heavy chain that is selected from the group of VH3 genes, more preferably the VH3 region that is encoded by any one of the VH3-23 and VH3-30 genes, most preferably the VH3-23 gene, encoded by the DNA sequence substantially as denoted by SEQ ID NO:25.

The linker region binding the first and the second binding regions encodes a peptide that is optimally long and flexible to enable simultaneous binding of both the first and other additional binding regions to their corresponding non-overlapping sites on the target protein(s), and should be substantially 10-100 amino acids in length. In one preferred embodiment, the linker comprises the amino acid sequence substantially as denoted by SEQ ID NO:17, and is encoded by the nucleic acid sequence substantially as denoted by SEQ ID NO:18.

The fusion polypeptide of the invention is preferably linked to an immunoglobulin scaffold. Thus, in this aspect, the nucleic acid molecule preferably further comprises a portion of a constant region of an immunoglobulin chain encoded by the nucleic acid sequence substantially as denoted by SEQ ID NO:26, which encodes human Cγ1 without CH1, starting immediately downstream to the first cysteine codon of the hinge region.

In one preferred embodiment, the invention provides a nucleic acid molecule, herein designated 632-3, having binding specificity for at least two different sites, at least one site being on the HIV envelope glycoprotein gp120 and the other site being either on said gp 120 protein or on the extracellular portion of human CD4, wherein the binding to said at least one site on said gp120 protein is independent of its binding to said other site on said gp120 protein or on said extracellular portion of human CD4, said chimeric protein essentially comprising:

(a) a first binding region comprising the two extracellular membrane distal domains of CD4, V1 and V2, encoded by the DNA sequence substantially as denoted by SEQ ID NO: 24;

(b) a second binding region comprising a binding site derived from a variable region of an antibody heavy chain encoded by the VH3-23 gene, substantially as denoted by SEQ ID NO: 25;

(c) a linker comprising the amino acid sequence substantially as denoted by SEQ ID NO: 17, and encoded by the nucleic acid sequence substantially as denoted by SEQ ID NO: 18; and

(d) a portion of a constant region of an immunoglobulin chain encoded by the DNA sequence substantially as denoted by SEQ ID NO: 26.

In another aspect, the invention provides an expression vector comprising a nucleic acid molecule as described above. In a further aspect, the invention provides host cells, preferably mammalian cells, transformed with said expression vectors.

In still another aspect, the invention provides a chimeric protein having binding specificity for at least two different sites, at least one site being on the HIV envelope glycoprotein gp120 and the other site being either on said gp120 protein or on the extracellular portion of human CD4, wherein the binding to said at least one site on said gp 120 protein is independent of its binding to said other site on said gp 120 protein or on said extracellular portion of human CD4, said chimeric protein essentially comprising:

(a) a first binding region comprising a soluble extracellular portion of human CD4;

(b) a second binding region that comprises a variable region of an antibody heavy chain, that is capable of being attached to an adjacent and non-overlapping site on the said gp120 protein or to a site on said extracellular portion of human CD4, and is capable of increasing the capacity of the said extracellular portion of human CD4 to interact with gp120 and to block the interaction of HIV with membranal CD4; and

(c) a linker region that physically connects both binding regions (a) and (b).

The first binding region of the chimeric protein is composed preferably of the two extracellular membrane distal domains of CD4, V1 and V2, encoded by the DNA sequence substantially as denoted by SEQ ID NO:24, and the second binding region is composed preferably of a variable region of an antibody heavy chain that is selected from the group of VH3 genes, more preferably the VH3 region that is encoded by any one of the VH3-23 and VH3-30 genes, most preferably the VH3-23 gene, encoded by the DNA sequence substantially as denoted by SEQ ID NO:25.

The linker region binding the first and the second binding regions is preferably a peptide that is optimally long and flexible to enable simultaneous binding of both the first and other additional binding regions to their corresponding non-overlapping sites on the target protein(s), and should be substantially 10-100 amino acids in length. In one preferred embodiment, the linker comprises the amino acid sequence substantially as denoted by SEQ ID NO: 17, and is encoded by the nucleic acid sequence substantially as denoted by SEQ ID NO: 18.

The fusion polypeptide of the invention is preferably linked to an immunoglobulin scaffold. Thus, in this aspect, the chimeric protein preferably further comprises a portion of a constant region of an immunoglobulin chain encoded by the nucleic acid sequence substantially as denoted by SEQ ID NO:26.

In one preferred embodiment, the invention provides a chimeric protein being the expression product of the clone herein designated 632-3, encoding a functional chimeric polypeptide having binding specificity for at least two different sites, at least one site being on the HIV envelope glycoprotein gp120 and the other site being other on said gp120 protein or on the extracellular portion of human CD4, wherein the binding to said at least one site on said gp120 protein is independent of its binding to said other site on said gp120 protein or on said extracellular portion of human CD4, said chimeric protein essentially comprising:

(a) a first binding region comprising the two extracellular membrane distal domains of CD4, V1 and V2, encoded by the DNA sequence, substantially as denoted by SEQ ID NO: 24;

(b) a second binding region comprising a binding site derived from a variable region of an antibody heavy chain encoded by the VH3-23 gene, substantially as denoted by SEQ ID NO: 25;

(c) a linker comprising the amino acid sequence substantially as denoted by SEQ ID NO: 17, and encoded by the nucleic acid sequence substantially as denoted by SEQ ID NO: 18; and

(d) a portion of a constant region of an immunoglobulin chain encoded by the DNA sequence substantially as denoted by SEQ ID NO: 26.

The invention further provides pharmaceutical compositions comprising a chimeric protein as defined above, for the prevention and treatment of HIV infection.

There is also provided a method for neutralizing and inhibiting HIV virus replication and infectivity in a subject, comprising administering to said subject an effective amount of a chimeric protein of the invention.

### BRIEF DESCRIPTION OF THE FIGURES

FIG. 1 depicts pBJ1-Neo-based cassettes of constructs according to the invention containing the intact human Cγ1 gene (56-1), the human Cκ gene (62-1), the human Cλgene (171), the intact human Cμ gene (803-6), the human Cγ1ΔCH1 gene (C308) and the mouse CμΔCH1 gene (812-1), for stable and transient expression of antibody chains and immunoligands. SRα=promoter; shaded boxes=exons. Restriction sites: H=HindIII; N=NotI; R=EcoRI; X=XhoI.

FIG. 2 schematically depicts predicted homodimeric structures of a native Ig molecule, 611-2 (VH3), and 632-3 (CD4-VH3) products. Each box represents an immunoglobulin or immunoglobulin-like domain.

FIG. 3 is a graph showing inhibition of binding of the CD4 immunoligand, the protein product of construct 611-2 (according to Example 2) to recombinant gp120 (rgp120),by the MAb Leu3a, known to bind to human CD4 V1 domain and to inhibit gp120 binding.

FIG. 4 shows binding of four products to rgp120 (2 nm of each as tested by direct ELISA on plastic immobilized rgp120). The products tested were the expression products of plasmids 630-8 (V1-VH3), 631-2 (VH3-V1), 632-3 (CD4-VH3) and 633-3 (VH3-CD4) and compared with the products of plasmids 614-2 (CD4 (V1+V2)) and 611-2 (VH3), which constitute the separate components.

FIG. 5 shows ELISA analysis of the specificity of gp120 binding by the chimeric protein CD4-VH3 (expressed by plasmid 632-3), against the specificity of its two constituents VH3 (expressed by plasmid 614-2) and CD4 (expressed by plasmid 611-2).

FIG. 6 shows an immunoblot of the protein products of plasmids 611- and 632-3, following polyacrylamide gel electrophoresis (PAGE), using Coomassie blue staining. Lanes 1-3=PAGE under non-reduced conditions; lanes 4-9=reduced conditions; lanes 1,7=human IgG1; lanes 2,5,6=611-2 (various concentrations); lanes 3,8,9=632-3 (various concentrations); lane 4=protein size marker.

### DETAILED DESCRIPTION OF THE INVENTION

The life cycle of animal viruses is characterized by a series of events that are required for the productive infection of the host cell. The initial step in the replicative cycle is the attachment of the virus to the cell surface which is mediated by the specific interaction of the viral attachment protein to receptors on the surface of the target cell. The pattern of expression of these receptors is largely responsible for the host range and tropic properties of viruses. The interaction of the viral attachment protein with cellular receptors therefore plays a critical role in infection and pathogenesis of viral diseases and represents an important area to target the development of anti-viral therapeutics.

Cellular receptors may comprise all of the components of membranes, including proteins, carbohydrates, and lipids. Identification of the molecules mediating the attachment of viruses to the target cell surface has been made in a few instances. The most extensively characterized viral receptor protein is CD4.

In humans, CD4 is also the target of interaction with the human HIV. HIV primarily infects helper T lymphocytes, monocytes, macrophages and dendritic cells that express surface CD4. Infection by HIV results in loss of helper T lymphocytes, which is one marker of the progress of HIV infection. The depletion of these cells is probably an important cause of the loss of immune function which results in the development of the human acquired immunodeficiency syndrome (AIDS). In contrast to helper T lymphocytes, other CD4+ cells, notably dendritic cells, monocytes and macrophages, are chronically infected by HIV. They produce virus over a long period of time and appear to be major reservoirs of HIV in vivo.

The initial phase of the HIV replicative cycle involves the high affinity interaction between gp120, the larger subunit of HIV exterior envelope glycoprotein (env), and cellular CD4. Several lines of evidence demonstrate the requirement of this interaction for viral infectivity. In vitro, the introduction of a functional cDNA encoding CD4 into human cells which do not express CD4 is sufficient to render otherwise resistant cells susceptible to HIV infection. In vivo, viral infection appears to be restricted to cells expressing CD4. Binding of HIV gp120 to cell surface CD4 induces a conformational change in gp120, which exposes, or stabilizes a second binding site to the virus co-receptor, primarily one of the two chemokine receptors CCR5 or CXCR4 (see, for example, WO 00/55207). Chemokine receptor binding leads to shedding of gp120 and to fusion of viral and target cell membranes, which is mediated by the smaller subunit of env, gp41. Fusion results in the introduction of the viral nucleocapsid into the target cell cytoplasm.

The present invention utilizes domains derived from native CD4 receptor molecules for creation of neutralizing chimeric proteins. Such domains, in their soluble forms, are often the molecules of choice when effects such as inhibition of harmful receptor-ligand interactions or virus neutralization are required, since they are directed exactly at the receptor-binding structure on the ligand. Furthermore, the present invention demonstrates improved biological function of the chimeric proteins, which results from the augmented affinity, compared with the single domains.

Thus, as a first aspect, the invention relates to an isolated nucleic acid sequence coding for a functional chimeric protein. This chimeric protein has binding specificity for at least one site on a target protein and to another site located either on the target protein or on the chimeric protein itself. Binding of the chimeric protein of the invention to the one site on the target protein is independent of its binding to any other site on said target protein or on the chimeric protein itself.

The chimeric protein of the invention essentially comprises:

a. a first binding region that recognizes a site on the target gp120 protein, this first binding region comprising an extracellular portion of human CD4;

b. at least one additional binding region that is capable of being attached either to an adjacent and non-overlapping site on the target gp120 protein or to a site on the chimeric protein itself, said at least one additional binding region comprising a binding site derived from a variable region of an antibody heavy chain; and

c. linker regions that physically connect said binding regions.

In one specifically preferred embodiment, the invention provides a nucleic acid molecule coding for a chimeric protein, said chimeric protein comprising:

a. a first binding region that recognizes a site on the target gp120 protein, this first binding region comprising the V1 and V2 domains of human CD4;

b. a second binding region that is capable of being attached either to an adjacent and non-overlapping site on the target gp120 protein or to a site on the chimeric protein itself, said at least one additional binding region comprising a binding site derived from the variable VH3 region of an antibody heavy chain; and

c. a linker region that physically connects both binding regions (a) and (b).

The nucleic acid molecule of the invention is an isolated biological component, meaning nucleic acids prepared by recombinant expression in a host cell or by PCR as well as chemically synthesized nucleic acids.

Chimeric protein is a fusion protein made of heterologous segments, which are naturally not normally fused in the same manner. Thus, the fusion product of CD4 V1 and V2 domains and the VH3, linked by peptidic linkers, is a continuous protein molecule having sequences fused by typical peptide bonds, typically made as a single translation product and exhibiting properties derived from each source peptide.

Chimeric proteins having multiple binding specificities are proteins that have at least two domains fused together, each domain comprising an independent binding region capable of forming a specific complex with a corresponding site in a target protein. In the present invention, the chimeric protein is composed of a soluble extracellular portion of human CD4 and a variable region of an antibody heavy chain, and one binding site is on gp 120 as the target protein of CD4 and the other binding site may be on the gp120 protein or on the CD4 portion of the chimeric protein itself. The two domains are genetically fused together, in that nucleic acid molecules that encode each protein domain are functionally linked together, for instance by a linker oligonucleotide, thereby producing a single chimera-encoding nucleic acid molecule. The translated product of such a chimera-encoding nucleic acid molecule is the chimeric protein. The chimeric protein is said to have “specific binding affinity” to a molecule or peptide, if it is capable of specifically reacting with that molecule or that peptide. By “functional” it is meant being capable of specifically binding to the target protein and inducing the neutralization and inactivation of any virus expressing the target protein.

The second binding region is said to be “capable of being attached” to an adjacent and non-overlapping site on said target protein, if this binding region displays “non-conventional” binding properties to said site in the target protein. The use of the term “non-conventional” binding encompasses binding due, for example, to “superantigen” binding ability or to adhesiveness or stickiness properties of said binding region to a certain site on the target protein. By “adjacent” it is meant any distance between the non-overlapping different sites on a target protein that can be specifically recognized simultaneously, by their corresponding binding regions of the chimeric protein of the invention. The distance between the adjacent sites on the target protein should be in such length and topography that allows the recognition of each site by the binding domains linked by the linker region. The different sites on the target protein may be located in tandem one to another, or alternatively may be located in distal regions on the target protein. As mentioned before, besides the possibility that the second binding region binds to the gp120 target protein, the invention also encompasses the possibility that the second component binds to the CD4 portion of the chimeric protein itself, this binding being independent of the binding of CD4 to the target gp120 protein.

In another preferred embodiment, the present invention relates to a chimeric protein as defined above that further comprises the constant region of an immunoglobulin chain, and therefore the creation of an antibody-like molecule. The generation of an antibody-like molecule is achieved by fusing the two different and independent binding sites as described above, to the constant region of a human IgG heavy chain, lacking CH1 domain, wherein two such constant regions homodimerize to form an Fc structure. Addition of the Fc portion has considerable advantages. Thus, the properties of immunoglobulins make them a suitable “backbone” for these CD4-VH3-based chimeric protein molecules, and confers on the chimeric proteins of the invention the ability to induce complement activation and the ability to mediate antibody-dependent cytotoxicity.

Immunoglobulins, or antibodies, are the antigen-binding molecules produced by B lymphocytes which comprise the humoral immune response. The basic unit of an immunoglobulin molecule consists of two identical heavy (H) chains and two identical light (L) chains. The amino-terminus of each chain contains a region of variable amino acid sequence (variable region—V). The variable regions of the heavy and light chains interact to form two antigen-binding sites. The carboxy-terminus of each chain contains a region of constant amino acid sequence (constant domain—C). The light chain contains a single constant domain, whereas the heavy chain constant domain is subdivided into four separate domains (CH1, hinge, CH2, and CH3). The heavy chains of immunoglobulin molecules are of several types, including μ, δ, γ, α and ε. The light chains of immunoglobulin molecules are of two types, either kappa (κ) or lambda (λ). Within the individual types of heavy and light chains exist subtypes which may differ in effector function.

These fusion proteins retain various effector functions of immunoglobulin molecules, such as Fc receptor binding, cell-mediated transfer via a Fc receptor-dependent mechanism and complement activation. Moreover, the CD4-immunoglobulin chimeras have a much longer half-life in vivo and increased solubility.

The constant regions of the antibody cloned and used in the chimeric immunoglobulin-like molecule may be derived from any mammalian source. The constant regions may be complement binding or may present antibody-dependent cell-mediated cytotoxicity (ADCC) activity.

As mentioned before, in a preferred embodiment, the first binding region of the chimeric protein of the invention is derived from the CD4 molecule. CD4 is Cluster of Differentiation factor 4, a T-cell surface protein that mediates interaction with the MHC class II molecule. CD4 also serves as the primary receptor site for HIV on T-cells during HIV infection. CD4 is a nonpolymorphic, lineage-restricted cell surface glycoprotein that is a member of the immunoglobulin gene superfamily.

Mature CD4 has a relative molecular mass (Mr) of 55 kilodaltons and consists of an amino-terminal 372 amino acid extracellular domain containing four tandem immunoglobulin-like regions denoted V1-V4, followed by a 23 amino acid transmembrane domain and a 38 amino acid cytoplasmic segment. The amino-terminal immunoglobulin-like V1 domain bears 32% homology with kappa light chain variable domains. Three of the four immunoglobulin-like domains contain a disulphide bond (V1, V2 and V4), and both N-linked glycosylation sites in the carboxy-terminal portion of the molecule are utilized.

Molecules that are derived from CD4 include fragments of CD4, generated either by chemical (e.g. enzymatic) digestion or genetic engineering means. Such a fragment may comprise one or more of the entire CD4 protein domains (for example, extracellular domains V1, V2, V3 and V4), as defined in the immunological literature, or a portion of one or more of these well-defined domains. For instance, a binding molecule or binding domain derived from CD4 would comprise a sufficient portion of the CD4 protein to mediate specific and functional interaction between the binding fragment and a native or viral binding site of CD4. One such binding fragment includes both the V1 and V2 extracellular domains of CD4 (CD4 V1+V2), though smaller fragments may also provide specific and functional CD4-like binding. The gp120-binding site has been mapped to V1 of CD4.

The term “CD4-derived molecules” also encompasses analogs (non-protein organic molecules), derivatives (chemically functionalized protein molecules obtained starting with the disclosed protein sequences) or mimetic (three-dimensionally similar chemicals) of the native CD4 structure, as well as protein sequence variants or genetic alleles, that maintain the ability to functionally bind to a target molecule.

In a specifically preferred embodiment, the isolated nucleic acid sequence of the invention encodes a chimeric protein that comprises as a first binding region, the two extracellular membrane distal domains of CD4, V1 and V2.

It has recently been discovered that patients infected with HIV were deficient in B cells expressing VH3 genes [Berberian et al., Blood 78:175-179 (1991)]. VH3 is the largest Ig heavy chain variable region gene family. Indeed, the VH3 family consists of 40 or more functional and non-functional related gene segments.

Acquired immunodeficiency syndrome (AIDS) is characterized by dysfunction and depletion of immune cells, including CD4 T-cells and B-cells. CD4 T-cells are selectively depleted by a chain of events that initiates with the binding of the human immunodeficiency virus (HIV) to the CD4 molecule via the gp120 viral envelope glycoprotein. The mechanism by which virus infection ultimately leads to depletion of this cellular population is unknown.

In another specifically preferred embodiment, the second binding-site is derived from a variable region of an antibody heavy chain.

The variable regions of both heavy and light chains show considerable variability in structure and amino acid composition from one antibody molecule to another, whereas the constant regions show little variability. The term “variable” as used herein refers to the diverse nature of the amino acid sequences of the antibody heavy and light chain variable regions. Nevertheless, the use of the heavy-chain variable region VH of an antibody molecule in the present invention is either as a superantigen binding domain or as a generally adhesive (‘sticky’) domain, or as a domain which can pair with the VL-like V1 domain of CD4, and the functional properties of antigen-binding of the antibody are not utilized in the present invention.

More specifically, the variable region of an antibody heavy chain may be selected according to the present invention from the group of VH3 genes. Preferably, the VH3 is encoded by any one of VH3-23 and VH3-30 genes. Most preferably, the second binding-site is derived from the VH3-23 gene. It is to be appreciated that the present invention further encompasses the possibilities that said binding region derived from the VH3-23 gene is attached either to a site on the gp120 target protein due to its adhesiveness or stickiness properties or to a site on the V1 portion of the CD4 molecule thus stabilizing a higher affinity conformation of the CD4 molecule for the target gp120 protein.

“Linker” as used herein is a peptide, usually between two and 150 amino acid residues in length that serves to join two protein domains in a multi-domain fusion protein. Examples of specific linkers can be found, for instance, in Hennecke et al. [Protein Eng. 11:405-410, (1998)] and U.S. Pat. Nos. 5,767,260 and 5,856,456. Depending on the domains being joined, and their eventual function in the fusion protein, linkers may be from about 10 to about 100 amino acids in length, though these limits are given as general guidance only. The tendency of the fusion proteins to form specific and non-specific multimeric aggregations is influenced by linker length [Alfthan et al., Protein Eng. 8:725-731, (1998)]. Thus, shorter linkers will tend to promote multimerization, while longer linkers tend to favor maintenance of monomeric fusion proteins. Aggregation can also be minimized through the use of specific linker sequences, as demonstrated in U.S. Pat. No. 5,856,456.

According to the invention, the linker connecting both binding components should be substantially flexible and long enough to enable simultaneous binding of both the first and second binding regions to their corresponding non-overlapping sites on the target protein or on the target protein and on the chimeric protein itself, but rigid and short enough so as to prevent overfreedom in the simultaneous binding, which may result in reduced effect. Linkers of about 25-100 Å, or about 15-100 amino acid residues in length, are examples of suitable linkers. Specific examples of linkers include, but are not limited to, one or more occurrences of the 17-amino acid sequence represented by SEQ ID NO:17. For instance, the invention encompasses nucleic acid sequences coding for chimeric proteins wherein the two binding domains are functionally and physically linked by the nucleic acid sequence represented by SEQ ID NO:18.

Another preferred embodiment of the invention relates to an expression vector comprising a nucleic acid molecule coding for a functional chimeric protein of the invention. The term “expression vectors”, as used herein, encompasses plasmids, viruses, bacteriophage, integratable DNA fragments, and other vehicles, which enable the integration of DNA fragments into the genome of the host. Expression vectors are typically self-replicating DNA or RNA constructs containing the desired gene or its fragments, and operably linked genetic control elements that are recognized in a suitable host cell and effect expression of the desired genes. These control elements are capable of effecting expression within a suitable host. Generally, the genetic control elements can include a prokaryotic promoter system or a eukaryotic promoter expression control system. Such system typically includes a transcriptional promoter, an optional operator to control the onset of transcription, transcription enhancers to elevate the level of RNA expression, a sequence that encodes a suitable ribosome binding site, RNA splice junctions, sequences that terminate transcription and translation and so forth. Expression vectors usually contain an origin of replication that allows the vector to replicate independently of the host cell.

A vector may additionally include appropriate restriction sites, antibiotic resistance or other markers for selection of vector containing cells. Plasmids are the most commonly used forms of vector but other forms of vectors which serve an equivalent function and which are, or become, known in the art, are suitable for use herein. See, e.g., Pouwels et al. Cloning Vectors: a Laboratory Manual (1985 and supplements), Elsevier, N.Y.; and Rodriquez, et al. (eds.) Vectors: a Survey of Molecular Cloning Vectors and their Uses, Buttersworth, Boston, Mass. (1988), which are incorporated herein by reference.

The term “operably linked” is used herein for indicating that a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

In general, such vectors contain in addition specific genes, which are capable of providing phenotypic selection in transformed cells. The use of viral expression vectors to express the genes coding for the polypeptides of the present invention are also contemplated.

As disclosed herein, recombinant expression vectors have been prepared which, when used to transfect mammalian host cells, permit the secretion of foreign protein outside the cytoplasmic membrane of the host cell. One preferred example of a recombinant plasmid of the invention is the plasmid herein designated 632-3 (see Table 3 herein).

Another aspect of the invention relates to a host cell transformed with any one of the expression vehicles of the present invention. “Host cell” as used herein refers to cells which can be recombinantly transformed with vectors constructed using recombinant DNA techniques. Suitable host cells include prokaryotes, lower eukaryotes, and preferably, higher eukaryotes, e.g. mammalian cells. Prokaryotes include gram negative and gram positive organisms, e.g., E. coli and B. subtilis. Lower eukaryotes include yeast, e.g. S. cerevisiae and Pichia, and species of the genus Dictyostelium. Higher eukaryotes include mammalian cell lines, preferably monkey COS cells for transient expression and CHO and B myeloma cells for stable expression.

The vector is introduced into a host cell by methods known to those of skilled in the art. Introduction of the vector into the host cell can be accomplished by any method that introduces the construct into the cell, including, for example, calcium phosphate precipitation, microinjection, electroporation or transformation. See, e.g., Current Protocols in Molecular Biology, Ausuble, F. M., ed., John Wiley & Sons, N.Y. (1989). In a specifically preferred embodiment, the host cell of the invention is transformed with the 632-3 recombinant plasmid of the invention.

In a further aspect, the invention relates to a chimeric protein having binding specificity for at least two different sites, at least one of these sites being on the target gp I20 protein. It is to be appreciated that the chimeric protein of the present invention may be purified prior to use. The term purified does not require absolute purity; rather, it is intended as a relative term. Thus, for example, a purified chimeric protein preparation is one in which the chimeric protein is more enriched than the protein is in its generative environment, for instance within a cell or in a biochemical reaction chamber. In some embodiments, a preparation of chimeric protein is purified such that the chimeric protein represents at least 50% of the total protein content of the preparation.

One skilled in the art will understand that there are myriad ways to purify recombinant polypeptides, and such typical methods of protein purification may be used to purify the chimeric proteins of the invention. Such methods include, for instance, protein chromatography methods including ion exchange, gel filtration, HPLC, monoclonal antibody affinity chromatography and isolation of insoluble protein inclusion bodies after over production. In addition, purification affinity-tags, for instance a six-histidine sequence, may be recombinantly fused to the protein and used to facilitate polypeptide purification. A specific proteolytic site, for instance a thrombin-specific digestion site, can be engineered into the protein between the tag and fusion itself to facilitate removal of the tag after purification. Commercially produced protein expression/purification kits provide tailored protocols for the purification of proteins made using each system. See, for instance, the QIAexpress™ expression system from QIAGEN (Chatsworth, Calif.) and various expression systems provided by INVITROGEN (Carlsbad, Calif.). Where a commercial kit is employed, the manufacturer's purification protocol is a particularly disclosed protocol for purification of the protein. For instance, proteins expressed with an amino-terminal hexa-his tag can be purified by binding to nickel-nitrilotriacetic acid (Ni—NTA) metal affinity chromatography matrix (The QIA expressionist, QIAGEN, 1997).

In a further aspect, the invention relates to a pharmaceutical composition for the prevention or treatment of HIV infection. The chimeric proteins of the invention may be administered directly to the subject to be treated, or it may be desirable to administer to the subject a pharmaceutical composition comprising the chimeric protein of the invention and a pharmaceutically acceptable carrier, adjuvant or diluent. Therapeutic formulations may be administered in any conventional dosage formulation. The preparation of pharmaceutical compositions is well known in the art and has been described in many articles and textbooks, see e.g., Remington's Pharmaceutical Sciences, Gennaro A. R. ed., Mack Publishing Company, Easton, Pa., 1990, and especially p 1521-1712 therein.

The pharmaceutical compositions may be formulated in unit dosage form, suitable for individual administration of precise dosages. The dose ranges for the administration of the chimeric protein of the invention are those which are large enough to produce the desired effect whereby the symptoms of HIV infection are ameliorated. The amount of active compound administered will be dependent on the subject being treated, the severity of the affliction, and the manner of administration, and is best left to the judgment of the prescribing clinician.

In yet another aspect, the invention relates to the use of a chimeric protein of the invention for the manufacture of a medicament for the prevention and treatment of HIV infection.

The chimeric proteins of the invention, for instance CD4-VH3, are particularly useful in the prevention of HIV infection during or immediately after exposure to HIV (e.g., mother/infant transmission, post-exposure prophylaxis, and as a topical inhibitor). In such instances, one or more doses of the chimeric protein are administered before or soon after the triggering event. To prevent or ameliorate mother/infant transmission of viral infection, for instance, it may be beneficial to administer the chimeric protein to the mother intermittently throughout pregnancy, and/or immediately before or following delivery, and/or directly to the newborn immediately after birth. Post-exposure prophylactic treatments may be particularly beneficial where there has been accidental exposure (for instance, a medically related accidental exposure), including but not limited to a contaminated needle-stick or medical exposure to HIV-1 contaminated blood or other fluid.

The invention further relates to a method for neutralizing and inhibiting HIV virus replication and infectivity in a mammalian subject comprising administering to a subject in need, an effective amount of a chimeric protein of the invention.

The invention further provides methods for neutralizing and inhibiting HIV by inactivating viral gp120 with a chimeric protein of the invention. Binding of a viral or recombinant gp120 protein to soluble CD4 or lymphocyte CD4 can also be blocked and/or prevented by contacting the gp120 protein with a chimeric protein of the invention.

Introduction of the chimeric molecules by gene therapy or passive DNA immunization may also be contemplated, for example, using naked DNA, retroviruses, adeno-associated virus (AAV) or other means to introduce the genetic material encoding the fusion proteins into suitable target tissues. In this embodiment, the target tissues having the cloned genes of the invention may then produce the fusion protein in vivo.

The following examples are representative of techniques employed in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

Materials

Host cells: COS7—an SV40-transformed fibroblastoid cell line of Green African Monkey kidney origin; CHO—a fibroblastoid cell line of Chinese hamster ovary origin; SP2/0—a B cell myeloma of BALB/c mouse origin.

Vectors: pBJ1-Neo [Lin, A. Y., et al. (1990) Science 249:677-679]. This bifunctional vector potentiates both high-yield transient expression in COS cells due to the presence of the required SV40 sequences and selection of stably-transfected cells due to the Neor gene. Gene expression is driven by the extremely efficient and highly versatile SRa promoter, immediately downstream to which genes can be easily cloned via a useful multiple cloning site (MCS).

Experimental Procedures

mRNA preparation: Performed using the Oligotex Direct mRNA Mini Kit—Qiagen, according to manual.

RT-PCR: First strand synthesis was performed by using 5 μl mRNA; 1× AMV RT buffer (Promega); 50 pmole primer; 1 mM each dNTPs; 1 μl Rnasin (Promega); 5 units AMV RT (Promega); ddw to 20 μl. 95° C. 5 min.; 42° C. 60 min., 52° C. 30 min.

PCR: 10 μl First strand; 50 pmole each primer; 1×PCR buffer (Promega) 2.5 units Taq polymerase (Promega); ddw to 50 μl.

94° C. 5 min.; 56° C. 2 min.; 74° C. 2 min.; 94° C. 1 min; 56° C. 2 min.; 74° C. 2 min.; last 3 steps repeated 28 times; 94° C. 1 min.; 56° C. 2 min.; 74° C. 5 min.

Transfection: Was performed by electroporation method. 4×106 cells at exponential growth were collected, resuspended in 800 μl of their growth medium and transferred to an electroporation cuvette. 20 μg of either native or linear plasmid DNA was added and the mixture was incubated 1 min. Electric shock was then applied with an Easyject instrument (EquiBio) at 350V, 750 μF, ∞ resistance. Cells were immediately diluted in their growth medium and plated in 96 well tissue culture plates at 1×106 and 3×106 cells per plate. For stable transfection G418 was added to the culture medium 24-48 hours later, at 2 mg/ml.

ELISA: 100 μl of 2 μg/ml rgp120 (IIIB, MN, Ba-L) or 2-5 μg/ml capturing antibody in coating buffer (carbonate-bi-carbonate pH 9.6) were adsorbed onto a well of an ELISA plate. Tissue culture supernatants or purified protein samples were added at serial dilutions, and, depending on reagents, assay was developed with HRP-conjugated polyclonal goat antibodies against human IgG (Fc) human IgM, mouse IgM or mouse IgG (Fc).

### Example 1

Construction of the Expression Systems

The panel of polypeptide chains that were designed in the present invention were expressed in mammalian expression systems. The configuration of choice for presenting the chimeric molecules of the invention was as homodimeric immunoligands. These homodimeric immunoligands are based on truncated antibody heavy chains, not requiring light chain for proper assembly and secretion [Hayden, M. S., et al. (1993) Tumor Immunobiology: A Practical Approach, IRL Press, p245-261; Chamow, S. M., & Ashkenazi, A., (1996) Trends Biotech. 14:52-60]. In some instances, full human antibody molecules were expressed. Mammalian expression of antibodies or antibody-like products usually yields high level of very stable proteins which are easy to detect and often display improved functions as compared with such reagents produced in bacteria. For quick primary assay, transient expression of the constructs of the invention was performed in COS7 cells. CHO or SP2/0 myeloma cells were used for stable expression, when a large amount of products was required.

The cloning vector used in the present invention was pBJ1-Neo. All the genetic constructs include introns between the stretches encoding the V-like domains and the genes for the constant (C) region of the Ig chain. In many mammalian expression systems, introns have been shown to increase product yields, and, in addition, they render cloning more facile and flexible. To ensure proper splicing of pre mRNA of the constructed genes, they all include a consensus donor splice site at their downstream end, provided with the PCR primers. All expression cassettes were produced with upstream XhoI and downstream NotI restriction sites for modular, positional cloning.

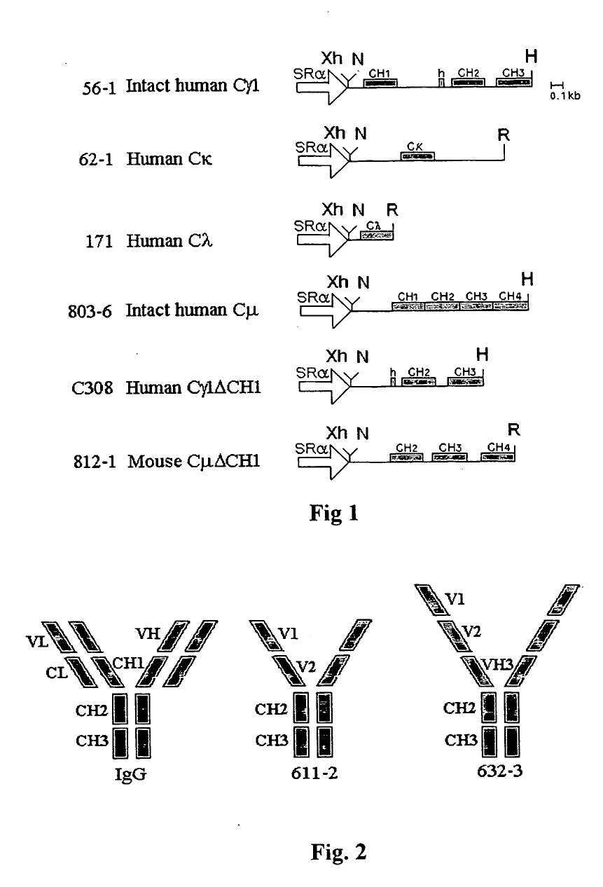

All together, six mammalian expression cassettes were constructed, which harbor antibody heavy or light chain constant region genes and unique XhoI and NotI cloning sites for gene segments encoding binding domains. All six cassettes are schematically illustrated in FIG. 1.

Cassette 56-1: The human Cγ1 gene was PCR-amplified from a genomic clone, using the upstream primer 24884: GCGGCCGCTAAGGTGAGGCAGGTG, herein denoted as SEQ ID NO:1, corresponding to positions 51-58 in GenBank accession (GBA) Z17370 and containing a NotI restriction site, and the downstream primer 202330: AAGCTTGCCGGCCGTCGCAC herein denoted as SEQ ID NO:2, covering positions 1810-1827 in the same GBA, with a HindIII site. The resulting fragment was inserted with NotI+HindIII into the pBJ1-Neo MCS.

Cassette 62-1: The full human CK gene was removed from a genomic clone as an XbaI/BamHI fragment, inserted into pBluescript KS II-(Stratagene,=KS), removed with NotI+EcoRI and cloned into pBJ1-Neo MCS.

Cassette 171: This vector was generated at XTL (Rehovot, Israel) and contains the full human Cλ2 gene from position 9425 to 9964 in GBA X51755.

Cassette 803-6: Poly A RNA was prepared from human peripheral blood cells. First strand synthesis was performed with an oligo dT primer, followed by nested RT-PCR. The upstream primer of the internal set was 29411: CCCCTGCAGGGAGTGCATCCGCCCCAACC herein denoted as SEQ ID NO:3, covering positions 1-21 in GBA X57086, with a PstI site. The downstream primer was 26700: GGGAAGCTTCAGTAGCAGGTGCCAGC herein denoted as SEQ ID NO:4, positions 1345-1362 in the same GBA with a HindIII site. An intronic sequence had to be supplemented upstream of the Cμ coding sequence. It was derived from human Cγ1 CH1-hinge intron. The upstream primer was 25657: GCGGCCGCCAGCACAGGGAGGGAGG, herein denoted as SEQ ID NO:5, positions 514-521 in GBA Z17370, with a NotI site. The downstream primer was 16657: GGGCTCTGCAGAGAGAAG, herein denoted as SEQ ID NO:6, positions 884-901, with a PstI and an acceptor splice site. The two PCR products were inserted in one step into pBJ1-Neo.

Cassette C308: A gene fragment encoding human Cγ1 without CH1, starting immediately downstream to the first cysteine codon of the hinge region was prepared with primer 32191: GCGCTGCAGACAAAACTCACACATGCCCACCG, herein denoted as SEQ ID NO:7, 911-934 in GBA Z17370, with a PstI site, and primer 23671: GCGAAGCTTGCCGGCCGTCGCAC, herein denoted as SEQ ID NO: 8, 1810-1817 with a HindIII site. The construct was provided with the same intronic sequence described above, and prepared similarly in one step. This construct is herein denoted as SEQ ID NO:26.

Cassette 812-1: Genomic mouse Cμ gene without CH1 was PCR-amplified from a genomic clone with primer 28390: GCGGCGGCCGCCAAACCCTCCCAGCAGG, herein denoted as SEQ ID NO:9, positions 332-349, GBAV00818 with a NotI site, and primer 27503: CGCGAATTCGCCTGG TTGAGCGCTAGC, herein denoted as SEQ ID NO:10, positions 1877-1894, with an EcoRI site. The product was cloned into pBJ1-Neo MCS.

### Example 2

Cloning and Expression of Human CD4

Poly A RNA was prepared from Jurkat cells (a human CD4 T cell leukemia), and the V1+V2 segment of CD4 was RT-PCR cloned, encompassing amino acids 1-178 of the mature protein [Maddon, P. J., et al, (1985) Cell 42:93-104. See correction: Littman D. R., et al. (1988) cell 55:541]. The upstream primer was 34116: CGCGCGCGGCCGCTCGAGGCCACAATGAACCGGG, herein denoted as SEQ ID NO:1 1, positions 68-85 in GBA M 12807, with an XhoI site. The downstream primer was 4283: GCGGCGGCCGCACTTACCTAGCACCAC GATGTCTATTTTGAA, herein denoted as SEQ ID NO: 12, positions 658-682 followed by a splice donor site and a NotI site. The product was inserted into cassette C308, producing clone 611-2, and into cassette 812-1, producing clone 813-1.

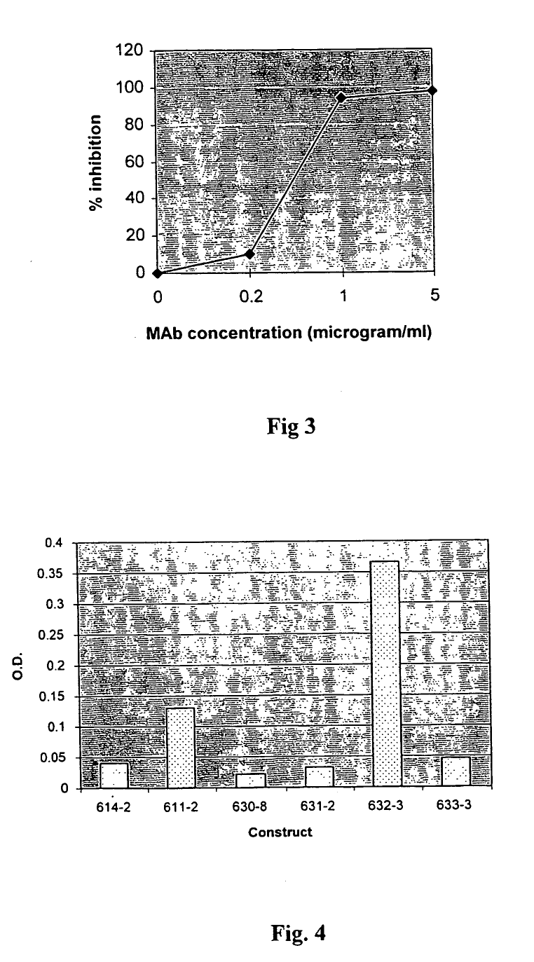

This DNA was transfected into COS7 cells, and culture supernatant was collected 96 hours later. ELISA experiments could detect the secretion several μg/ml of human IgG1, with considerable binding of recombinant gp120 (rgp120). This DNA was then used to generate stable transfectants of CHO cells, and the protein product was purified from a high producer on a Protein A column. The specificity of gp120 binding was confirmed with the MAb Leu3a (Becton Dickinson), known to bind to human CD4 V1 domain and inhibit gp120 binding. Results of this inhibition experiment are shown in FIG. 3.

### Example 3

Cloning and Expression of VH3 Genes

A VH3 domain, encoded by either VH3-23 or VH3-30, is a likely candidates to be linked to the membrane-distal portion of CD4, since as described in the background it have been implicated in gp120 binding.

It was then chosen to incorporate to the genetic construct of the invention a germline-encoded sequence of either VH3-23 or VH3-30, supplemented with an arbitrary CDR3 coding stretch and an FR4 (framework region), corresponding to the joining (J) segment. In an attempt to minimize the size of the protein product, the inventors have focused on the VH domain alone rather than on an intact Fv. In the absence of a VL domain, VH domains may exhibit non-specific binding, most likely due to exposure of CDR2 hydrophobic residues, which are normally buried in the VH/VL interface. It has been shown that VH domains can mimic those of camel antibodies, which entirely lack light chains, following the introduction of several point mutations into their CDR2 [Davies, J., & Riechmann, L., (1995) Biotechnology 13:475-479]. However, natural, non-paired human VH domains have displayed high degree of antigen specificity when expressed on phage [Cai, X., & Garen, A., (1997) Proc. Natl. Acad. Sci. USA 94:9261-9266]. Interestingly, and most relevant to the present invention, four out of the six VH domains analyzed in this study were encoded by VH3-23, or DP-47 [see Tomlinson, I. M., et al. (1992) J. Mol. Biol. 227:776-798 for the DP nomenclature of human germline V genes; VH3-30 is DP-49].

In addition, it was reasoned that weak, non-specific binding may in fact, contribute to overall affinity enhancement, providing yet another contact region following formation of the complex mediated by the principal, higher affinity binding sites. Such an effect can form the basis for a more general mode of affinity enhancement.

As starting materials, three cloned human VH genes were obtained in a rearranged configuration encoded by the relevant VH3 members, isolated from a semi-synthetic phage display scFv library [kindly provided by Dr. A. Nissim of the Hebrew University of Jerusalem: Nissim, A., et al. (1994) EMBO J. 13:692-698]. All VH genes in this library are linked to a single human Vλ gene. These genes were DP-47 (VH3-23) from an anti 2-phenyl-5-oxazolone (phOx) scFv (clone 16); DP-47 from an anti-keyhole limpet hemocyanin (KLH) scFv (clone 24); DP-49 (VH3-30) from an anti-thyroglobulin scFv (clone 37). These genes were cloned into expression cassettes in three formats: (i) First, as a part of a gene encoding the entire scFv, with VH at the amino terminus. (ii) Second, the VH3 segment alone. (iii) Third, as a part of an intact human IgG1/λ antibody.

(i) Cloning as a scFv

Two primers were designed. The upstream one, 8104, contained an XhoI site; the full leader sequence of DP-47 (not included in the phage clone), originally designated VH-26 [Matthyssens, G & Rabbitts, T. H., (1980) Proc. Natl. Acad. Sci. USA 77:6561-6565]; the first 6 codons of DP-47, with a T-G substitution at position 13, as in DP-49 [see Tomlinson, I. M., et al. (1992) J. Mol. Biol. 227:776-798]: CTCGAGATGGAGTTTGGGCTGAGCTGGCTTTTTCTTGTGGCTATTTTAAAA GGTGTCCAGTGTGAGGTGCAGCTGGTGGAG, herein denoted as SEQ ID NO: 13. The downstream primer, 36134, corresponds to the 3′ part of the human Jλ3 gene, GBA X51755 positions13675 to 13676, with a donor splice site and a NotI restriction site: GCGGCCGCACTTACCTAGGACGGTCAGCTTGGTCCC, herein denoted as SEQ ID NO:14.

(ii) Cloning as Single VH Domain

Primer 8104 (SEQ ID NO:13) was used together with primer 5153, which is a degenerate primer corresponding to the 3′ part of human JH gene segments according to GBA J00256, with a donor splice site and a Not I site: GCGGCGGCCGCACTCACCTGAGGAGACGGTGACCAGGGT(A/G/C)CCTTGG CCCCA, herein denoted as SEQ ID NO: 15.

DNA sequence of the amplified VH3 genes was determined, and all PCR products were cloned into C308 via the modular XhoI+NotI sites.

(iii) Cloning as an Intact Human IgG1/λ

All VH genes generated obtained for expression as single domains were inserted into the 56-1 cassette (see Example 1). The Vλ gene from the phage library present in all clones was amplified with the upstream primer 8703: GCGCTCGAGATGGCCTGGACCCCTCTCTGGCTCACTCTCCTCACTCTTTGCA TAGGTTCTGTGGTTTCTTCTGAGCTGACTCAGGAC, herein denoted as SEQ ID NO:16, corresponding to the intact Vλ leader sequence and the first seven codons of the mature λ light chain [Frippiat, J. -P., et al, (1990) Nucl. Acids. Res 18:7134] with an XhoI site and primer 36134 (SEQ ID NO:14). This product was inserted into the 171 cassette (see Example 1), creating clone 639-6.

Table 1 below summarizes the VH3 clones produced for expression.

TABLE 1
VH3 clones

| Clone No. | VH3 clone | Format | Expression Cassette |
|---|---|---|---|
| 603-1 | 16 | Intact Ig (+ 639-6) | 56-1 |
| 604-3 | 24 | Intact Ig (+ 639-6) | 56-1 |
| 605-3 | 37 | Intact Ig (+ 639-6) | 56-1 |
| 613-2 | 16 | Single domain | C308 |
| 614-2 | 24 | Single domain | C308 |
| 615-2 | 37 | Single domain | C308 |
| 78-2 | 37 | scFv | C308 |
| 79-1 | 16 | scFv | C308 |
| 80-6 | 24 | scFv | C308 |

Plasmid DNA was transfected into COS7 cells and supernatants were assayed by ELISA for hIgG1 and for the antigens phOx coupled to bovine serum albumin (phOx-BSA), KLH, and BSA alone. All proteins were secreted at several μg/ml of human IgG1 or human IgG1/λ, but all bound BSA alone at a level indistinguishable from their anticipated specific binding. Furthermore, binding to plastic immobilized rgp120 showed no specificity as well.

In parallel, a set of experiments to establish gp120 superantigen (SA) binding to human antibodies was performed. These included commercial human sera, commercially purified human IgM and IgG, and VH3-enriched full human IgM libraries were prepared in the present invention in COS7 cells. To construct these libraries a RT-PCR on poly A RNA prepared from peripheral blood cells was performed, using a human Cμ specific primer for cDNA synthesis, and primers 8104 (SEQ ID NO: 13) and 5153 (SEQ ID NO: 15), the former enabling enrichment for VH3 products. The heavy chain cassette was 803-6 and a number of light chain genes cloned into either the 62-1 or 171 light chain cassette were used in co-transfection for production of full IgM antibodies. Primary ELISA screening of transfectant supernatants was performed with anti-human IgM reagents. For gp120 SA binding experiments, rgp120 from several HIV-1 laboratory strains was used. All samples which exhibited gp120 binding were also positive for a panel of irrelevant proteins including BSA, KLH gelatin and milk. In addition, a later report analyzing gp120 SA-binding human serum antibodies [Townsley-Fuchs, J., et al. (1996) J. Clin. Invest. 98:1794-1801], described a gp120 SA-binding serum IgM titer which is much lower than that originally described [Berberian, L., et al. (1993) Science 249:1588-1591], requiring serum concentration of at least 1% for detection by direct ELISA. Moreover, this later study also reports that gp120 SA binding is masked by commonly used ELISA blocking agents such as albumin and milk. These findings and this report rendered the adequacy of gp120 SA properties for the inventors purpose questionable; nevertheless, the DP-47 anti-KLH VH clone was chosen for further manipulations.

### Example 4

Cloning and Expression of CD4-VH3 Fusion Products

Two configurations were tested in parallel. In the first, CD4 was at the amino terminus of the polypeptide, followed by VH3; in the second, these binding sites were linked at the inverted order. The linker peptide chosen for this task is the peptide 218 [Whitlow, M., & Filpula, D., (1992) Tumor Immunobiology, Oxford University Press,. pp:279-291], of the sequence:

Gly-Ser-Thr-Ser-Gly-Ser-Gly-Lys-Pro-Gly-Ser-Gly-Glu-Gly-Ser-Thr-Lys-Gly

herein denoted by SEQ ID NO: 17, and encoded by the nucleotide sequence:

GGCTCTACTTCCGGTTCAGGAAAGCCCGGGAGT GGTGAAGGTAGCACT AAAGGT, herein denoted by SEQ ID NO: 18. A SmaI restriction site is underlined.

First Configuration: CD4(V1+V2)−VH3

CD4 V1+V2 segment was PCR amplified with primers 34116 (containing an XhoI site and a new downstream primer, 6055, corresponding to the same CD4 positions as in primer 4283 (SEQ ID NO: 12) and the 5′ part of linker 218, including the SmaI site:

CGCCCCGGGCTTTCCTGAACCGGAAGTAGAGCCAGCTAGCACCACGATGT CTATTTTGAA herein denoted by SEQ ID NO:19.

VH3 from clone 24 was amplified with a new upstream primer, 5159, containing the 3′ part of linker 218 with the SmaI site and the first 6 codons of VH3 as in primer 8104:

GCGCCCGGGAGTGGTGAAGGTAGCACTAAAGGTGAGGTGCAGCTG GTGGAG, herein denoted by SEQ ID NO:20. The downstream primer was 5153 (SEQ ID NO: 15) with a NotI site.

Both PCR products were subcloned for DNA sequence determination and then cloned in one step into the C308 cassette, to produce clone 632-3 (CD4 V1+V2−VH3).

In addition, it was tested whether the requirement for CD4 V2 domain in the interaction with gp120 can be circumvented by linking VH3 in either end of V1 domain. For this purpose, two constructs in C308 have been assembled, in a similar design to that described for 633-3 and 632-3. In clone 630-8 the V1 gene segment is linked upstream to the VH3 gene (V1-VH3), and in clone 631-2 the order is inverse (VH3-V1).

Second Configuration: VH3-CD4(V1+V2)

VH3 from clone 24 was amplified with the upstream primer 22164: GCGCTGGAGA TGGAGTTTTGGGC, herein denoted by SEQ ID NO:21, 2, which corresponds to the 5′ end of primer 8104 (SEQ ID NO: 13), with an XhoI site, and with the downstream primer 5160, encoding the 3′ part of the VH gene joined in frame to the 5′ half of linker 218 including the SmaI site:

CGCCCCGGGCTTTCCTGAACCGGAAGTAGAGCCTGAGGAGACGGTGACCA G, herein denoted by SEQ ID NO:22.

The CD4(V1+V2) segment was amplified with primer 5445:

GCCCCCGGGAGTGGTGAAGGTAGCACTAAAGGTCAGGGAAAGAAAGTGG TGCTG, herein denoted by SEQ ID NO:23, with the 3′ part of linker 218 including the SmaI site joined in-frame to the seven first codons of the mature CD4 protein, and primer 4283 with a NotI site.

Both PCR products were similarly subcloned for DNA sequence determination and then cloned in one step into the C308 cassette, producing clone 633-3 VH3-CD4 V1+V2).

Plasmids 630-8, 631-2, 632-3 and 633-33 were transfected into COS7 cells. Supernatants were first assayed by ELISA with anti-human IgG1 reagents against commercial human IgG1 and concentrations of the secreted products were determined. Next, product concentrations were calibrated and tested by direct ELISA on plastic-immobilized rgp120, together with products 614-2 (CD4 V1+V2) and 611-2 (VH3) which constitute the separate components of the two hybrids. Results are presented in FIG. 4.

Similar results have been obtained in several experiments and consistently demonstrated approximately 3-fold enhancement in the signal of 632-3 compared with 611-2. On the other hand, the inverse arrangement, represented by the product of clone 633-2, had shown a marked decrease in signal compared with 611-2, and was not pursued further. The same was true for the two combinations lacking CD4 V2.

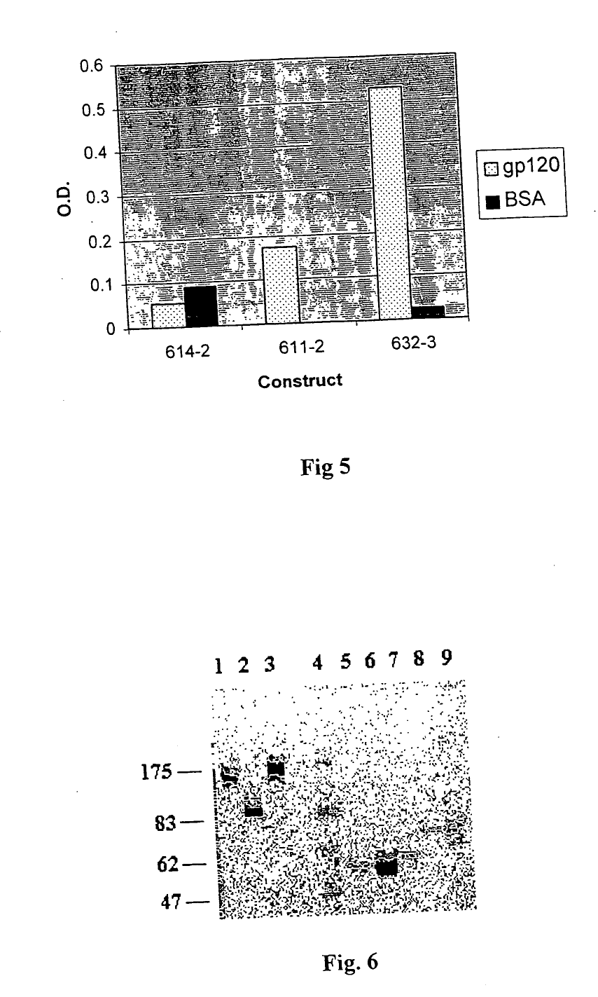

An ELISA analysis of the specificity of gp120 binding is shown in FIG. 5. VH3 alone showed no specificity of binding to rgp120. This non-specific binding was markedly decreased in product 632-2 and, in some experiments, was non-detectable.

Plasmids 611-2 and 632-3 were then transfected into SP2/0 B myeloma or CHO cells. Stably-transfected cells were selected with G418, and high producers were expanded. Protein products were purified from transfectant supernatants on a protein A column, analyzed first by ELISA and then in an immunoblot following polyacrylamide gel electrophoresis (PAGE), using Coomassie blue staining, as shown in FIG. 6. The molecular weight of intact human IgG1 is approximately 150 kD, and of its heavy chain monomer seen in the immunoblot is 55 kD. The expected size of product 611-2 is 104 kD (monomer of 52 kD) and that of product 632-3 is 132 kD (66 kD), as summarized in Table 2 below.

TABLE 2
Deduced molecular weight of protein products

| | | Deduced |
|---|---|---|
| Product No. | Protein Structure | dimer size (kD) |
| 611-2 | CD4(V1 + V2)-hIgG1 | 104 |
| 632-3 | CD4(V1 + V2)-linker-VH3-hIgG1 | 132 |

### Example 5

Testing Products in HIV-1 Neutralization Assays

To test the products of the invention in HIV-1 neutralization, the protein products listed in Table 2 above were purified on protein A columns, filter-sterilized and accurately calibrated for human IgG1 concentration. All HIV neutralization experiments were performed in the laboratory of Dr. David Montefiori, Center for AIDS Research, Duke University Medical School, Durham, N.C., USA.

To evaluate neutralization of two laboratory HIV-1 strains by the chimeric proteins of the invention, the first experiment presented in Table 3 was designed. The parameter ID50 is defined as the dose (product concentration) required to protect 50% of MT-2 cells (an HTLV-1-immortalized human T cell line) from virus-induced cell killing as measured by neutral red uptake. This value corresponds to approximately 90% reduction in synthesis of p24 (the viral core protein, as monitored by a p24 immunoassay described by Zhou and Montefiori (1997), J. Virol. 71:2512-2517).

TABLE 3
Neutralization of HIV-1 laboratory strain

| ID50 (nM) against: | | |
|---|---|---|
| Product No. | HIV-1 IIIB | HIV-1 MN |
| 611-2 | 0.79 | <0.41 |
| 632-3 | 0.46 | 0.15 |

As shown in Table 3, construct 611-2 (VH3) activity was comparable to results previously reported for CD4 immunoadhesins [see Capon, D. J., & Ward, R. H. R., (1991) Annu. Rev. Immunol. 9:649-678 and references therein]. However, construct 632-3 (CD4 V1+V2−VH3) showed 1.5-3 fold improvement and higher neutralization capacity than 611-2. Since the protein products assemble as truncated human IgG1 heavy chain homodimers, differences in neutralization potential are unlikely to reflect mere avidity effects.

In the next experiment, the protein products were screened for neutralization in PMBC (peripheral blood mononuclear cells) of primary HIV-1 isolates from clade C (the global HIV-1 epidemic comprises infections from at least 9 genetically distinct and geographically related subtypes, or clades, of the virus). Concentrations were not equalized, but rather one tenth of the original concentration was used. Results are shown in Table 4.

TABLE 4
Screening of clade C primary isolates in PMBC
% reduction in p24 against strain:

| | Conc. | DU151 | DU179 | DU368 | DU422 |
|---|---|---|---|---|---|
| Product No. | (nM) | (R5) | (R5/X4) | (R5) | (R5) |
| 611-2 | 890 | 63 | 78 | 53 | 9 |
| 632-3 | 330 | 74 | 88 | 68 | 23 |

Values are % reduction in p24 synthesis relative to sample (virus control). Concentrations of p24 in virus control wells were 12.8, 7.4, 47.1 and 3.3 ng/ml for the 4 isolates from left to right

This experiment implies stronger neutralization by the chimeric protein of the invention as compared with CD4, but it was not yet followed by a dose-response curve. Clade C isolates are relatively resistant to soluble CD4 (Dr. D. Montefiori, personal communication to the inventor).

The third neutralization experiment was performed similarly in PMBC with clade B primary isolates, and its results are presented in Table 5. Both constructs showed marked inhibition with most isolates in the concentration used.

TABLE 5
Screening of clade B primary isolates in PMBC
% reduction in p24 against strain:

| | Conc. | | | | | |
|---|---|---|---|---|---|---|
| Product No. | (nM) | GV | JR-FL | P15 | PVO | Bal |
| 611-2 | 890 | 88 | 100 | 94 | 96 | 100 |
| 632-3 | 330 | 96 | 100 | 99 | 96 | 100 |

Values are % reduction in p24 synthesis relative to sample (virus control). Concentrations of p24 in virus control wells were 6.9, 10.1, 22.8, 4.1 and 6.5 ng/ml for the 5 isolates from left to right.

Next, titration experiments were performed with the primary isolates Ba1 and JR-FL of clade B, and ID90 values are given in Table 6.

TABLE 6
ID90 values in PMBC of two clade B primary isolates

| ID90 (nM) | | |
|---|---|---|
| Product No. | Bal | JR-FL |
| 611-2 | 5.3 | 8.2 |
| 632-3 | <0.4 | 0.4 |

Samples were assayed at 3-fold dilutions starting at the concentrations used for the screening experiments. Values are the concentrations (nM) in which p24 production was reduced 90% relative to no sample (virus control). Concentrations of p24 in virus control were 13.7 ng/ml (Ba1) and 26.1 ng/ml (JR-FL).

Construct 632-3 displayed an impressive >13-20 fold increase compared with 611-2, as showed by the results.

The encouraging results obtained with the lade B primary isolates and the laboratory strains indicate that the chimeric proteins of the invention can be used for prevention and treatment of HIV infection.

1. A nucleic acid molecule encoding a functional chimeric protein having binding specificity for at least two different sites, at least one site being on the HIV envelope glycoprotein gp120 and the other site being either on said gp120 protein or on the extracellular portion of human CD4, wherein the binding of said chimeric protein to said at least one site on said gp120 protein is independent of its binding to said other site on said gp120 protein or on said extracellular portion of human CD4, said chimeric protein essentially comprising:
(a) a first binding region comprising a soluble extracellular portion of human CD4;
(b) a second binding region comprising a variable region of an antibody heavy chain, that is capable of being attached to an adjacent and non-overlapping site on the said gp120 protein or to a site on said extracellular portion of human CD4, and is capable of increasing the capacity of the said extracellular portion of human CD4 to interact with gp120 and to block the interaction of HIV with membranal CD4; and
(c) a linker region that physically connects both binding regions (a) and (b).

2. The nucleic acid molecule according to claim 1, wherein said first binding region comprises the two extracellular membrane distal domains of human CD4, V1 and V2, encoded by the DNA sequence substantially as denoted by SEQ ID NO:24.

3. The nucleic acid molecule according to claim 1 or 2, wherein said second binding region comprises a variable region of an antibody heavy chain that is selected from the group of VH3 genes.

4. The nucleic acid molecule according to claim 3, wherein said VH3 region is encoded by any one of the VH3-23 and VH3-30 genes.

5. The nucleic acid molecule according to claim 4, wherein the VH3 is encoded by the VH3-23 gene, encoded by the DNA sequence substantially as denoted by SEQ ID NO:25.

6. The nucleic acid molecule according to any one of claims 1 to 5, wherein the linker region encodes a peptide that is optimally long and flexible to enable simultaneous binding of both the first and the second binding regions to their corresponding non-overlapping sites on the target gp120 protein or on the gp120 protein and on the extracellular portion of human CD4.

7. The nucleic acid molecule according to claim 6, wherein said linker is substantially 10-100 amino acids in length.

8. The nucleic acid molecule according to claim 7, wherein said linker comprises the amino acid sequence substantially as denoted by SEQ ID NO:17, and is encoded by the nucleic acid sequence substantially as denoted by SEQ ID NO:18.

9. The nucleic acid molecule according to any one of claims 1 to 8, wherein said chimeric protein further comprises a portion of a constant region of an immunoglobulin chain.

10. The nucleic acid molecule according to claim 9, wherein said portion of a constant region of an immunoglobulin chain is encoded by the nucleic acid sequence substantially as denoted by SEQ ID NO:26.

11. A nucleic acid molecule, herein designated 632-3, having binding specificity for at least two different sites, at least one site being on the HIV envelope glycoprotein gp120 and the other site being either on said gp120 protein or on the extracellular portion of human CD4, wherein the binding to said at least one site on said gp120 protein is independent of its binding to said other site on said gp120 protein on said extracellular portion of human CD4, said chimeric protein essentially comprising:
(a) a first binding region comprising the two extracellular membrane distal domains of CD4, V1 and V2, encoded by the DNA sequence substantially as denoted by SEQ ID NO: 24;
(b) a second binding region comprising a binding site derived from a variable region of an antibody heavy chain encoded by the VH3-23 gene, substantially as denoted by SEQ ID NO: 25;
(c) a linker comprising the amino acid sequence substantially as denoted by SEQ ID NO: 17, and encoded by the nucleic acid sequence substantially as denoted by SEQ ID NO: 18; and
(d) a portion of a constant region of an immunoglobulin chain encoded by the DNA sequence substantially as denoted by SEQ ID NO: 26.

12. An expression vector comprising a nucleic acid molecule according to any one of claims 1 to 11.

13. An expression vector according to claim 12, comprising the nucleic acid molecule of claim 11.

14. A host cell transformed with an expression vector according to claim 12.

15. A host cell transformed with an expression vector according to claim 13.

16. A chimeric protein having binding specificity for at least two different sites, at least one site being on the HIV envelope glycoprotein gp120 and the other site being either on said gp120 protein or on the extracellular portion of human CD4, wherein the binding of said chimeric protein to said at least one site on said gp120 protein is independent of its binding to said other site on said gp120 protein or on said extracellular portion of human CD4, said chimeric protein essentially comprising:
(a) a first binding region comprising a soluble extracellular portion of human CD4;
(b) a second binding region comprising a variable region of an antibody heavy chain, that is capable of being attached to an adjacent and non-overlapping site on the said gp120 protein or to a site on said extracellular portion of human CD4, and is capable of increasing the capacity of the said extracellular portion of human CD4 to interact with gp120 and to block the interaction of HIV with membranal CD4; and
(c) a linker region that physically connects both binding regions (a) and (b).

17. The chimeric protein according to claim 16, wherein said first binding region comprises the two extracellular membrane distal domains of human CD4, V1 and V2, encoded by the DNA sequence substantially as denoted by SEQ ID NO:24.

18. The chimeric protein according to claim 16 or 17, wherein said second binding region comprises a variable region of an antibody heavy chain that is selected from the group of VH3 genes.

19. The chimeric protein according to claim 18, wherein said VH3 region is encoded by any one of the VH3-23 and VH3-30 genes.

20. The chimeric protein according to claim 19, wherein the VH3 is encoded by the VH3-23 gene, encoded by the DNA sequence substantially as denoted by SEQ ID NO:25.

21. The chimeric protein according to any one of claims 16 to 20, wherein the linker region consists of a peptide that is optimally long and flexible to enable simultaneous binding of both the first and the second binding regions to their corresponding non-overlapping sites on the target gp120 protein or on the gp120 protein and on the extracellular portion of human CD4.

22. The chimeric protein according to claim 21, wherein said linker is substantially 10-100 amino acids in length.

23. The chimeric protein according to claim 22, wherein said linker comprises the amino acid sequence substantially as denoted by SEQ ID NO: 17, and is encoded by the nucleic acid sequence substantially as denoted by SEQ ID NO: 18.

24. The chimeric protein according to any one of claims 16 to 23, wherein said chimeric protein further comprises a portion of a constant region of an immunoglobulin chain.

25. The chimeric protein according to claim 24, wherein said portion of a constant region of an immunoglobulin chain is encoded by the nucleic acid sequence substantially as denoted by SEQ ID NO:26.

26. A chimeric protein having binding specificity for at least two different sites, at least one site being on the HIV envelope glycoprotein gp120 and the other site being either on said gp120 protein or on the extracellular portion of human CD4, wherein the binding to said at least one site on said gp120 protein is independent of its binding to said other site on said gp120 protein or on said extracellular portion of human CD4, said chimeric protein essentially comprising:
(a) a first binding region comprising the two extracellular membrane distal domains of CD4, V1 and V2, encoded by the DNA sequence substantially as denoted by SEQ ID NO: 24;
(b) a second binding region comprising a binding site derived from a variable region of an antibody heavy chain encoded by the VH3-23 gene, substantially as denoted by SEQ ID NO: 25;
(c) a linker comprising the amino acid sequence substantially as denoted by SEQ ID NO: 17, and encoded by the nucleic acid sequence substantially as denoted by SEQ ID NO:18; and
(d) a portion of a constant region of an immunoglobulin chain encoded by the DNA sequence substantially as denoted by SEQ ID NO: 26.

27. A pharmaceutical composition comprising a chimeric protein according to any one of claims 16 to 26, and a pharmaceutically acceptable carrier.

28. The pharmaceutical composition according to claim 27, for prevention and treatment of HIV infection.

29. A method for neutralizing and inhibiting HIV virus replication and infectivity in a subject, comprising administering to said subject an effective amount of a chimeric protein according to any one of claims 16 to 26.
